(19) United States
(12) Patent Application Publication
(10) Pub. No.: US 2014/0205541 A1
(43) Pub. Date: Jul. 24, 2014

(54) Method for Detection of Characteristics of Organ Fibrosis

(71) Applicant: Acuitas Medical Limited, Swansea (GB)

(72) Inventors: Timothy W. James, Santa Barbara, CA (US); Samantha Telfer, Newport (GB); Amanda Davies, Cardiff (GB); Lance W. Farr, Swansea (GB); Ryan Pathak, Caerphilli (GB); Kristin James, Santa Barbara, CA (US)

(73) Assignee: Acuitas Medical Limited, Swansea (GB)

(21) Appl. No.: 14/224,502

(22) Filed: Mar. 25, 2014

Related U.S. Application Data
(63) Continuation of application No. PCT/US2012/057207, filed on Sep. 26, 2012.
(60) Provisional application No. 61/539,276, filed on Sep. 26, 2011.

(51) Int. Cl. A61K 49/06

(52) U.S. Cl. CPC A61K 49/06
USPC 424/9.3

(57) ABSTRACT

The disclosed invention is a method for detecting indications of the presence of liver disease and other fibrotic diseases using a magnetic-resonance based technique for measuring fine tissue and bone textures. Specifically, the invention focuses on adaptations to this prior art to facilitate assessment of the presence and severity of liver disease, lung disease, and other fibrotic disease by measuring spatial wavelengths characteristic of the specific disease process across an areal cross-section through an organ. The results may be presented using a mapping technique. In this way, the resolution of MR is extended further than possible with current MR imaging, so as to be able to measure the fine scale structures and tissue changes that are known to be characteristic of the degenerative processes involved in the development of these diseases.

### CROSS-REFERENCE TO RELATED APPLICATIONS

This application is a continuation of International Application No. PCT/US2012/057207 filed Sep. 26, 2012 which claims the benefit of U.S. Provisional Patent Application No. 61/539,276 filed Sep. 26, 2011.

### BACKGROUND OF THE INVENTION

1. Field of the Invention

The present invention relates to the field of diagnostic assessment and monitoring of fibrotic diseases such as liver disease, lung disease, cystic fibrosis, intestinal fibrosis, pancreatic fibrosis, myelofibrosis, arthofibrosis, muscular dystrophy, renal fibrosis in kidney disease, and other diseases in which the attendant tissue and organ degeneration involves development of fibrotic structures, in clinical practice as well as in clinical and preclinical research.

2. Prior Art

Five and a half million people in the US are estimated to have CLD (Chronic Liver Disease) or its more developed form, liver cirrhosis, costing the US healthcare system some $1.6 billion annually, and accounting for approximately 27,000 deaths per year. In the United Kingdom liver disease is the fifth largest cause of deaths (see N. C. Henderson, S. J. Forbes, “Hepatic fibrogenesis: From within and outwith”, Toxicology, 254, 130-135, 2008). It is considered to be a major health concern in the rest of the world. Although there are many causes of liver disease, the most common feature for the majority of diseases is fibrosis, which can develop over many years. Advanced fibrosis can lead to cirrhosis, portal hypertension (reduction of blood flow through the liver) and reduced function or failure of the liver. Patient management in advanced disease is restricted to transplant, with an uncertain outcome, but often by this point development of carcinoma or other complications results in a dim prognosis. For this reason, diagnosing liver disease early on, when a range of management options are available, and the ability to monitor disease progression/regression in response to therapy are major needs in healthcare.

Although the underlying causes of liver disease may vary, fibrosis occurs in practically all variants of this disease. It is the main wound healing response to injury in the liver (see N. C. Henderson, S. J. Forbes, “Hepatic fibrogenesis: From within and outwith”, Toxicology, 254, 130-135, 2008) as it is in other fibrotic diseases that attack other organs, for example lung diseases. Liver fibrosis is defined as “the excessive accumulation of extracellular matrix proteins” (see R. Bataller and D. A. Brenner, “Liver Fibrosis” The Journal of Clinical Investigation, 115, 209-218, 2005). Generally fibrosis develops over many years. For a considerable period of time, up to a decade or more, patients may experience limited symptoms, until the fibrosis becomes particularly advanced (see S. L. Friedman, “Hepatic fibrosis-Overview”, Toxicology, 254, 120-129, 2008 and M. Pinzani, K. Rombouts, “Clinical Review: Liver fibrosis: from the bench to clinical targets” Digestive and Liver Disease, 36, 231-242, 2004). In the advanced stages a patient will suffer reduced liver function as a result of portal hypertension (reduction of blood flow through the liver) (see M. Pinzani, K. Rombouts, “Clinical Review: Liver fibrosis: from the bench to clinical targets” Digestive and Liver Disease, 36, 231-242, 2004 and S. L. Friedman, “Liver fibrosis—from bench to bedside”, Journal of Hepatology, 38, S38-S53, 2003).

Cirrhosis is the final stage of fibrosis, in which the liver function, architecture and appearance have been greatly altered to the point that liver failure is inevitable if nothing is done to reverse the course of the disease (see M. Pinzani, K. Rombouts, “Clinical Review: Liver fibrosis: from the bench to clinical targets” Digestive and Liver Disease, 36, 231-242, 2004, S. L. Friedman, “Liver fibrosis—from bench to bedside”, Journal of Hepatology, 38, S38-S53, 2003 and R. G. Wells, “Mechanisms of liver fibrosis: New insights into an old problem” Drug Discovery Today: Disease Mechanisms, 3, 4, 489-495, 2006). However, even in this advanced stage of the disease, up to 40% of patients may be asymptomatic (see S. L. Friedman, “Liver fibrosis—from bench to bedside”, Journal of Hepatology, 38, S38-S53, 2003). The development of cirrhosis in the liver also brings the increased risk of developing liver cancer (see N. C. Henderson, S. J. Forbes, “Hepatic fibrogenesis: From within and outwith”, Toxicology, 254, 130-135, 2008, R. Bataller and D. A. Brenner, “Liver Fibrosis” The Journal of Clinical Investigation, 115, 209-218, 2005, and M. Pinzani, K. Rombouts, “Clinical Review: Liver fibrosis: from the bench to clinical targets” Digestive and Liver Disease, 36, 231-242, 2004). Therefore, advanced fibrosis can obviously lead to high morbidity and death.

As is the case for most diseases, early detection allows the most options for disease management and the best prognosis. If caught early enough, liver disease is reversible. The main response to liver fibrosis is to treat or remove the underlying cause (see R. Bataller and D. A. Brenner, “Liver Fibrosis” The Journal of Clinical Investigation, 115, 209-218, 2005 and M. Pinzani, K. Rombouts, “Clinical Review: Liver fibrosis: from the bench to clinical targets” Digestive and Liver Disease, 36, 231-242, 2004) where possible. Very early on, this may be as simple as changes in lifestyle. Research directed at use of anti-fibrotic therapy and anti-inflammatory drugs have shown promising results. Other therapies under development are targeted at inhibiting various factors associated with fibrosis; these include—apoptosis of hepatocytes, activation/accumulation of myofibroblasts such as stellate cells, collagen production or promotion of degradation (see R. Bataller and D. A. Brenner, “Liver Fibrosis” The Journal of Clinical Investigation, 115, 209-218, 2005). In addition to the need in disease management, means of in vivo assessment of disease progression and regression is needed for monitoring as part of this therapy development.

Currently, diagnosis can be performed through liver function tests, imaging techniques, and biopsy. There are several standard staging systems associated with biopsy that define disease progression by five stages where generally: F0 is no fibrosis present, F1, fibrous tissue expanding around portal vessels; F2 fibrous tissue extending from the portal triads; F3 septa connecting central veins and portal triads; F4, cirrhosis—regenerated liver cells surrounded by fibrous septa (see S. C. Faria, K. Ganesan, I. Mwangi, M. Shiehmorteza, B. Viamonte, S. Mazhar, M. Peterson, Y. Kono, C. Santillan, G. Casola C. B. Sirlin, “MR Imaging of Liver Fibrosis: Current State of the Art”, RadioGraphics, 29, 1615-1635, 2009).

CLD (Chronic Liver Disease) results in a range of patterns of fibrotic tissue formation with intervening liver tissue showing relative hypertrophy in a micro and/or macroscopically nodular morphology that becomes more pronounced as the disease progresses (see M. Pinzani, K. Rombouts, “Clinical Review: Liver fibrosis: from the bench to clinical targets” Digestive and Liver Disease, 36, 231-242, 2004). In active hepatic inflammation, either in the setting of acute or acute on chronic inflammation, the “active” element relates generally to histological measures of inflammatory cell infiltrates and necrosis. Currently, biopsy remains the gold standard for quantified diagnosis of liver disease processes. A significant limitation of disease quantification by biopsy is that liver disease is characteristically non-uniform, both across the entire organ as well as also on a centimeter scale. Therefore, biopsy, which samples a few cubic millimeters from one location in the liver periphery, may misrepresent the general status of the liver. Dependence on liver biopsies may also be limiting access to care because clinicians and/or patients are reluctant to use an invasive, and often very painful, test with attendant potentially serious complication risks (such as bleeding, gallbladder puncture, etc.) and the usual recommendation that the patient spends several hours at the medical center for post-biopsy observation (see D. C. Rockey, S. H. Caldwell, Z. D. Goodman, R. C. Nelson and A. D. Smith, “Liver biopsy”, Hepatology, 49, 3, 1017-1044, 2009). Approximately 2-3% of patients undergoing liver biopsy require hospitalization for the management of an adverse effect (see T. Pasha, S. Gabriel, T Therneau, E. R. Dickson And K. D. Lindor, “Cost-Effectiveness of Ultrasound-Guided Liver Biopsy”, Hepatology, 27, 5, 1220-1226, 1998 and C H. Janes and K. D. Lindor, “Outcome of Patients Hospitalized for Complications after Outpatient Liver Biopsy”, Annals of Internal Medicine, 118, 2, 96-98, 1993). Thirty percent of patients experience significant pain during the procedure, and fatal complications have been reported in 0.01-0.3% of biopsied patients (see F. D. Srygley and K. Patel, “Noninvasive Assessment of Liver Fibrosis in Chronic Hepatitis C Infection”, Current Hepatitis Reports, 7, 164-172, 2008 and T. Gilmore, A. Burroughs, I. M. Murray-Lyon, R. Williams, D. Jenkins, A. Hopkins, “Indications, methods, and outcomes of percutaneous liver biopsy in England and Wales: an audit by the British Society of Gastroenterology and the Royal College of Physicians of London”, Gut, 36, 437-441, 1995). Therefore, only some 5% of patients at risk for liver fibrosis receive biopsy. The extracted tissue is analysed through histology, which takes time to perform; of more concern is the fact that the analysis is subjective, resulting in relatively large variation in diagnosis—can be as high as 35%, which can lead to misdiagnosis (see N H. Afdhal, “Biopsy or Biomarkers for Diagnosis of Liver Fibrosis?”, Clinical Chemistry 50, 8, 1299-1300, 2004 and D. C. Rockey, S. H. Caldwell, Z. D. Goodman, R. C. Nelson and A. D. Smith, “Liver biopsy”, Hepatology, 49, 3, 1017-1044, 2009). The ability to provide a non-invasive surrogate for biopsy, such as MR-based assessments, that can visualize the significant regions of the liver would be a major advance for both clinical practice and for developing therapeutics.

In clinical practice, after lifestyle evaluation, liver serum tests are indicated when symptoms dictate, to determine if chronic infection is present. A positive result indicates risk of hepatocellular carcinoma, so patients are referred for MR imaging with Gd contrast agent to highlight tumor formation. At this stage of assessment the ability to evaluate the degree of fibrotic development would provide a much-needed marker for determination of presence and stage of disease. However, due to resolution limits, current MR-imaging techniques have not been able to provide a reliable assessment for these purposes. A number of MR-based approaches have been attempted, but none has delivered a non-invasive automated or semi-automated diagnostic for quantifying the fine texture changes associated with CLD. One approach that has received significant development effort uses non-enhanced (see Mitchell D G, Navarro V J, Herrine S K, Bergin D, Parker L, Frangos A, et al. “Compensated hepatitis C: unenhanced MR imaging correlated with pathologic grading and staging”. Abdom Imaging 2007) or contrast enhanced MRI (CE-MRI) (see Semelka R C, Chung J J, Hussain S M, Marcos H B, Woosley J T, “Chronic Hepatitis: Correlation of Early Patchy and Late Linear Enhancement Patterns on Gadolinium—Enhanced MR Images with Histopathology Initial Experience”, Journal Of Magnetic Resonance Imaging, 13,385-391, 2001, Aguirre D A, Behling C A, Alpert E, Hassanein T I, Sirlin C B. Liver fibrosis: noninvasive diagnosis with double contrast material-enhanced MR imaging. Radiology 2006; 239:425-437 and Martin D R, Seibert D, Yang M, Salman K, Frick M P. Reversible heterogeneous arterial phase liver perfusion associated with transient acute hepatitis: findings on gadolinium-enhanced MRI. J Magn Reson Imaging 2004; 20:838-842) in which liver morphology has been used as a component of this analytical approach (see Martin D R, Semelka R C. Magnetic resonance imaging of the liver: review of techniques and approach to common diseases. Semin Ultrasound CT MR 2005; 26:116-131). A dual contrast technique combining use of an ultra-small paramagnetic iron-oxide reticuloendothelial uptake agent with a gadolinium-based agent has been reported to depict hepatic fibrosis (HF) (see S. C. Faria, K. Ganesan, I. Mwangi, M. Shiehmorteza, B. Viamonte, S. Mazhar, M. Peterson, Y. Kono, C. Santillan, G. Casola C. B. Sirlin, “MR Imaging of Liver Fibrosis: Current State of the Art”, RadioGraphics, 29, 1615-1635, 2009). Unfortunately the iron-oxide agents are no longer readily available clinically and the morphological approach is qualitative and not amenable to automation.

Elastography, either MRI or ultrasound based has recently been adopted for detecting and quantifying hepatic fibrosis by inference from measures of tissue stiffness, which is related to the level of fibrosis (see Yin M, Chen J, Glaser K J, Talwalkar J A, Ehman R L. Abdominal magnetic resonance elastography. Top Magn Reson Imaging 2009; 20:79-87). These approaches, and in particular the magnetic resonance based technique, are developmental, require considerable expertise, require additional hardware and setup or a separate exam with a skilled operator, and are cumbersome for the patient. It has been used to stage fibrosis in a range of liver diseases including chronic HCV, primary biliary cirrhosis, recurrence of hepatitis C in transplanted livers and chronic hepatitis B (see M. Yang, D. R. Martin, N. Karabulut and M. P. Frick, “Comparison of MR and PET Imaging for the Evaluation of Liver Metastases”, Journal of Magnetic Resonance Imaging, 17, 343-349, 2003).

Computed Tomography (CT) can provide excellent images of livers with cirrhosis and lesions, i.e. where the morphology of the liver has been greatly altered. In addition it can image problems associated with liver disease that occur outside the liver itself—ascites, splenomegaly (enlarged spleen) (see S. C. Faria, K. Ganesan, I. Mwangi, M. Shiehmorteza, B. Viamonte, S. Mazhar, M. Peterson, Y. Kono, C. Santillan, G. Casola C. B. Sirlin, “MR Imaging of Liver Fibrosis: Current State of the Art”, RadioGraphics, 29, 1615-1635, 2009). However, its sensitivity to the early stages of fibrosis is much lower and fibrosis is not clearly visible—mainly due to motion from respiratory and cardiac cycles (see F. D. Srygley and K. Patel, “Noninvasive Assessment of Liver Fibrosis in Chronic Hepatitis C Infection”, Current Hepatitis Reports, 7, 164-172, 2008). Imaging can be enhanced using contrast agents whilst breath hold techniques can allow some enhancement with scans acquired during the different phases of blood flow, arterial and venous (see S. Bonekamp, I. Kamel, S. Solga and J. Clark, “Can imaging modalities diagnose and stage hepatic fibrosis and cirrhosis accurately?”, Journal of Hepatology, 50, 17-35, 2009). An additional disadvantage is that a patient is exposed to ionising radiation (x-rays), limiting the use of this modality for screening or longitudinal monitoring.

Positron emission tomography (PET) and single photon emission tomography (SPECT)—both techniques use radioactive tracers and therefore have the disadvantage of some radiation exposure. In addition, PET requires a cyclotron to be nearby. They provide images of function not structure and could be used evaluate the effects of disease but have limited spatial resolution. PET has been used to detect liver metastases with comparable results to MRI, although Yang et al (see M. Yang, D. R. Martin, N. Karabulut and M. P. Frick, “Comparison of MR and PET Imaging for the Evaluation of Liver Metastases”, Journal of Magnetic Resonance Imaging, 17, 343-349, 2003) found that the spatial resolution was limited and in addition it was more difficult to anatomically locate lesions in the liver. Therefore, they are rarely used in monitoring or diagnosing liver disease (see S. Bonekamp, I. Kamel, S. Solga and J. Clark, “Can imaging modalities diagnose and stage hepatic fibrosis and cirrhosis accurately?”, Journal of Hepatology, 50, 17-35, 2009).

Ultrasound encompasses many techniques that are used in diagnosing and monitoring liver disease. Ultrasound imaging is the most widely utilised image modality in clinical use for liver disease. Like CT it is successful in diagnosing cirrhosis but has variable and limited results with less advanced fibrosis. Reproducibility of results is also an issue, with variability between operators, machines, and physiological status of patients (see S. Bonekamp, I. Kamel, S. Solga and J. Clark, “Can imaging modalities diagnose and stage hepatic fibrosis and cirrhosis accurately?”, Journal of Hepatology, 50, 17-35, 2009).

Because of these various problems with current assessment techniques, a non-invasive method of fibrotic assessment allowing determination of disease presence and progression would facilitate efforts to treat this disease.

Lung disease is another pathology with attendant fibrotic response (see FIG. 4). Idiopathic pulmonary fibrosis (IPF) is the most common of more than 200 conditions generally grouped as interstitial lung diseases (ILD) (see Michiel Thomeer et.al, Clinical Use of Biomarkers of Survival in Pulmonary Fibrosis, Respiratory Research, 11:89, 2010). The cause of IPF, like many ILDs, is unknown, but the association of IPF with factors such as smoking and exposure to dust (see Talmadge E, King, Jr., Clinical Advances in the Diagnosis and Therapy of the Interstitial Lung Diseases, Am. J. Respir. Crit. Care Med., 172, 268-279, 2005), along with an increased incidence of pulmonary hypertension, suggests that the fibrosis associated with IPF is a result of physical injury to lung tissue (see Brett Ley et al, Clinical Course and Prediction of Survival in Idiopathic Pulmonary Fibrosis, Am. J. Respir. Crit. Care Med., 183, 431-440, 2011). IPF is also associated with an increased incidence of lung cancer.

In this pathology also, the earlier evidence of the disease can be detected, the more options are available for its management. Although the prognosis for IPF is poor and current therapies are generally ineffective, an improving understanding of the mechanism of IPF and interest in the development of new drugs offer hope for future success in treatment. A method for fast, accurate, non-invasive assessment and monitoring of IPF would therefore be of significant value.

A number of techniques have been evaluated for diagnosis of IPF, including serum biomarkers, high-resolution computed tomography (HRCT), PET, spirometry, walking tests and biopsy (see Michiel Thomeer et.al, Clinical Use of Biomarkers of Survival in Pulmonary Fibrosis, Respiratory Research, 11:89, 2010 and S. Bonekamp, I. Kamel, S. Solga and J. Clark, “Can imaging modalities diagnose and stage hepatic fibrosis and cirrhosis accurately?”, Journal of Hepatology, 50, 17-35, 2009). All have significant drawbacks. Serum biomarkers and PET provide only minimal diagnostic accuracy. Spirometry and walking tests provide some correlation with survival, but do not show statistical differences in therapy trials. HRCT has emerged as a clinical standard for diagnosis of IPF and can produce a semi-quantitative measure of fibrosis (see C. Isabella S. Silva et al, Nonspecific Interstitial Pneumonia and Idiopathic Pulmonary Fibrosis: Changes in Pattern and Distribution of Disease over Time, Radiology, 247 (1), 251-259, 2008), but it suffers from several significant drawbacks:

1) the AUC for HRCT is only about 0.6, so the combination of sensitivity and specificity is poor.

2) in a significant number of cases, the appearance of disease is atypical and cannot conclusively identify IPF (see N. Sverzelatti et al, High Resolution Computed Tomography in the Diagnosis and Follow-up of Idiopathic Pulmonary Fibrosis, Radiol. med., 115, 526-538, 2010).

3) HRCT requires thins slices and therefore significant radiation dose.

Biopsy is definitive but is often not an option as it is highly invasive and the health of the patient may not allow it (see Talmadge E, King, Jr., Clinical Advances in the Diagnosis and Therapy of the Interstitial Lung Diseases, Am. J. Respir. Crit. Care Med., 172, 268-279, 2005 and S. Bohla and J Schulz-Menger, “Cardiovascular Magnetic Resonance Imaging of Non-ischaemic Heart Disease: Established and Emerging Applications”, Heart, Lung and Circulation, 19, 117-132, 2010).

Moreover, it is well understood that IPF may exist in a “subclinical” state for an extended period prior to diagnosis because none of the current techniques has the capability for early detection (see Brett Ley et al, Clinical Course and Prediction of Survival in Idiopathic Pulmonary Fibrosis, Am. J. Respir. Crit. Care Med., 183, 431-440, 2011). There exists a clear need for a non-invasive tool to detect lung fibrosis in its early stages.

In addition to the liver and the lungs, most organs and tissues of the human body can be affected by fibrosis, the accumulation of extra-cellular material and/or replacement of normal tissue by extra-cellular material, mainly forms of collagen. Within the heart, myocardium in the right ventricle can be replaced by fibrotic-fatty tissue in arrhythmogenic right ventricular cardiomyopathy/dysplasia (ARVC/D) (see S. Bohla and J Schulz-Menger, “Cardiovascular Magnetic Resonance Imaging of Non-ischaemic Heart Disease: Established and Emerging Applications”, Heart, Lung and Circulation, 19, 117-132, 2010). Dilated cardiomyopathy (DCM) may occur because of a range of disorders (inflammatory, connective tissue and infiltrative) that lead to myocardial fibrosis where late-Gadolinium enhancement MRI can be used to identify its presence (see F. Rieder and C. Fiocchi, “Intestinal fibrosis in inflammatory bowel disease—Current knowledge and future perspectives”, Journal of Crohn's and Colitis, 2, 279-290, 2008). Most components of the digestive system—the upper and lower gastrointestinal tract and the accessory organs, can develop fibrosis. Similarly to the liver, inflammation in the intestine (as a result of inflammatory bowel disease) and ulcerative colitis result in fibrotic response (see F. Rieder and C. Fiocchi, “Intestinal fibrosis in inflammatory bowel disease—Current knowledge and future perspectives”, Journal of Crohn's and Colitis, 2, 279-290, 2008). Together, Crohn's disease and ulcerative colitis affect around 250,000 people in the UK. In the spleen, parenchymal and capsular fibrosis can occur following injury induced by a variety of chemicals (see A. W. Suttie, “Histopathology of the spleen”, Toxicologic Pathology, 34, 466, 2006), while pancreatic fibrosis, is a feature of chronic pancreatitis of various causes (see P. S. Haber, G. W. Keogh, M. V. Apte, C. S. Moran, N. L. Stewart, D. H. G. Crawford, R. C. Pirola, G. W. McCaughan, G. A. Ramm, J. S. Wilson, “Activation of Pancreatic Stellate Cells in Human and Experimental Pancreatic Fibrosis”, American Journal of Pathology, 155, 4, 1087-1095, 1999). The esophagus has also been observed to develop fibrosis in those suffering from eosinophilic esophagitis, and gastroesophageal reflux disease, one of the most common problems encountered in clinical practice today (see F. Rieder, P. Biancani, K. Harnett, L. Yerian, G. W. Falk, “Inflammatory mediators in gastroesophageal reflux disease: impact on esophageal motility, fibrosis and carcinogenesis”, American Journal of Physiology Gastrointestinal and Liver Physiology, 298, G571-G581, 2010).

Fibrosis can occur in the components of the musculoskeletal system—skeleton, joints, muscles, etc. In bones, primary and secondary myelofibrosis results in fibrotic tissue replacing bone marrow (see N. Srinivasaiah, M. K. Zia and V. Muralikrishnan, “Peritonitis in myelofibrosis: a cautionary tale”, Hepatobiliary Pancreat. Dis. Int. 9, 6, 651-653, 2010). The majority of joints can experience arthrofibrosis, described as restriction of movement due to the formation of intra-articular scar tissue as a result of some form of injury (see M. Martin J. Gillespie, J Friedland And K. E. Dehaven, “Arthrofibrosis: Etiology, Classification, Histopathology And Treatment”, Operative Techniques in Sports Medicine, 6, 2, 102-110, 1998). Fibrosis of the muscles, where the deposition of extracellular matrix progressively remodels, destroys and replaces normal tissue, is characteristic of virtually all neurodegenerative muscular diseases.

The urinary system is also subject to the development of fibrosis. Renal fibrosis occurs in almost every type of chronic kidney disease. Development of fibrosis is progressive and results in the necessity of dialysis or kidney transplantation. Extracellular matrix is deposited around the functional filtration units of the kidney (renal corpuscle) and in the interstitium surrounding the tubules, distorting the fine architecture of kidney tissues leading to collapse of renal parenchyma and loss of kidney function (see Y Liu, “Renal fibrosis: New insights into the pathogenesis and therapeutics”, Kidney International, 69, 213-217, 2006). Abnormal deposition of fibrous tissue within layers of the bladder wall leads to changes in the vesical volume which may contribute to renal fibrosis and failure. Fibrosis in the bladder occurs secondary to spina bifida in the pediatric population and results in severe morbidity (see P. D. Metcalfe, J. Wang, H. Jiao, Y. Huang, K. Hori, B. D. Moore, E. E. Tredget, “Bladder outlet obstruction: progression from inflammation to fibrosis”, BJU International, 106, 11, 1686-1694, 2010).

Due to the ubiquitous nature of fibrotic response attendant with disease onset and progression in a vast number of disease states, a low-cost, non-invasive assessment technique for tissue fibrosis would greatly improve patient outcomes, both directly, as well as by enabling longitudinal monitoring of response to therapy.

### BRIEF DESCRIPTION OF THE DRAWINGS

FIG. 1. a) Interleaved selectively excited internal volumes aligned with the coronal plane. b) Overlaid view of the prisms location in the right anatomical liver lobe, the reference image is of the slice in which the prism array lies.

FIG. 2. Histological samples demonstrating the progression of fibrosis development from grade 0 (no disease) to grade 4 (Cirrhosis).

FIG. 3. Wavelength spectra displaying highest intensity between a) 0.5 mm and 1 mm, b) 1 mm and 3 mm and c) 3 mm and 5 mm, shown alongside corresponding intensity maps for the same ranges. The specific region of map generated from the individual spectrum is indicated by the arrow.

FIG. 4. Histological samples displaying normal alveoli (A) and alveoli from an individual suffering from idiopathic pulmonary fibrosis (B).

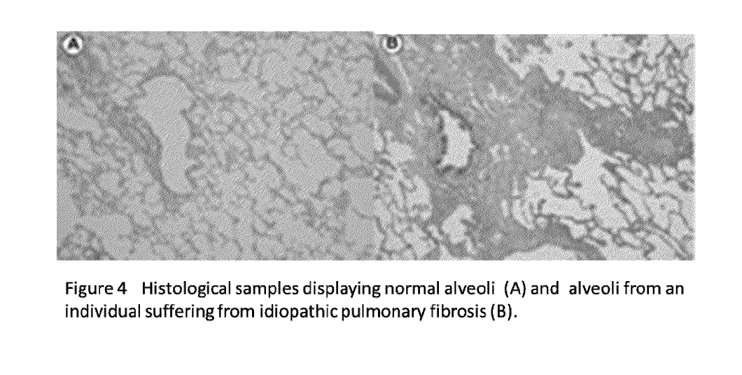

### DETAILED DESCRIPTION OF THE PREFERRED EMBODIMENTS

The current invention may be practiced, by way of example, by adaptations of the methods disclosed in U.S. Pat. No. 7,932,720, a magnetic resonance fine texture measurement technique, and U.S. Pat. No. 7,903,251, a technique to map fine structure characteristics over an area of interest in an organ, for assessment and monitoring of fibrotic diseases, specifically by measurement of targeted wavelength ranges characteristic of the tissue changes attendant with organ degeneration and recovery in specific diseases, and display of this measured information in such a manner as to allow assessment and monitoring of disease onset, progression and severity.

In order to define terminology for what follows, an internal volume in the anatomy of interest is excited by proper sequencing of magnetic field gradients and RF (Radio Frequency) pulses. Acquisition of the finely sampled 1D data is enabled by application of a readout gradient along a selected direction within the volume. Data acquisition is the acquiring of spatially-encoded MR echoes along an acquisition axis of a selectively-excited internal volume.

The inner volume can be defined in a multitude of shapes and sizes; as one example, by application of orthogonal magnetic gradients and subsequent application of two RF pulses of properly selected bandwidth, a rectangular prism-shaped volume can be excited. By application of a readout gradient, for example along the long axis of the prism, finely sampled echo data can be acquired along this axis. Although a rectangular prism is one possible volume with which to acquire data, many other “volumes” are possible.

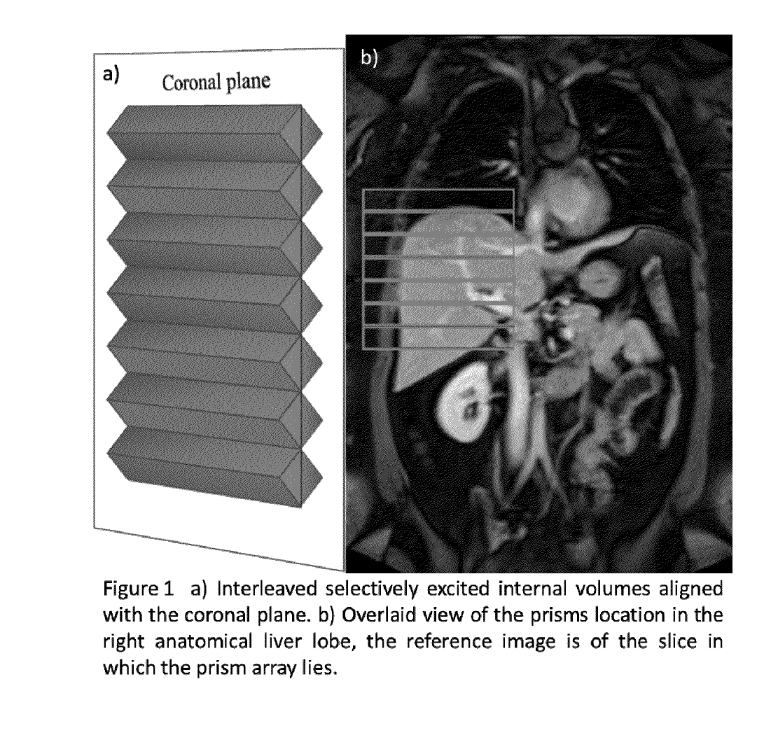

The readout gradient defines the direction of echo data acquisition. The term “readout gradient direction” may be used interchangeably with “acquisition axis” or “direction of data acquisition” or “data acquisition direction” or “acquisition direction” in the following. Additionally, to specify the volume of tissue within which the MR data is excited “selectively-excited inner volume”, “inner-volume”, and “acquisition volume” are also used interchangeably in the following. In the prior art technique to map fine structure characteristics over an area of interest in an organ, the areal coverage is obtained by interleaving several acquisition volumes in such a way that data is taken along the acquisition axis in each of the interleaved volumes within one data acquisition series (see FIG. 1).

The present invention consists of techniques for detecting onset and progression of liver disease and other fibrotic diseases through measurement of and mapping the structural wavelengths (also known as textural wavelengths), or other markers derived from the MR data, which are indicative of disease progression. Specifically, the techniques facilitate the assessment of the fine scale fibrotic structures attendant in liver disease progression, as well as in progression of a range of fibrotic diseases including those listed in the prior art section above. Further, these adaptations are designed to allow implementation of the techniques as a low-cost, non-invasive, fast add-on to a routine MR exam, such as might be ordered to rule out the risk of HCC (Hepatocellular Carcinoma) in suspected liver disease.

The prior art magnetic resonance fine texture measurement techniques, which may be used to obtain a spectrum of structural (textural) wavelengths in a tissue (the invention applies to spectrums of textural wavelengths, no matter how obtained), provides the resolution capability to detect changes in fine texture representative of the early stages of liver disease (chronic or acute) and other disease states. Combined with a technique for mapping structural wavelength data across an areal cross-section, which may be the prior art technique, and with suitable adaptation to derive markers from the structural wavelengths appropriate for assessment of a particular disease, the combined techniques can be applied to the assessment of early and later stages of disease.

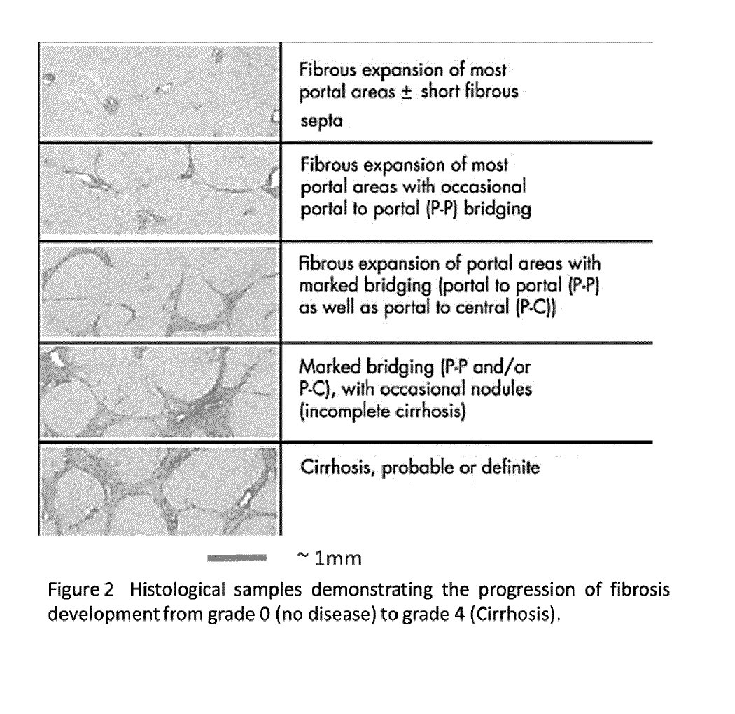

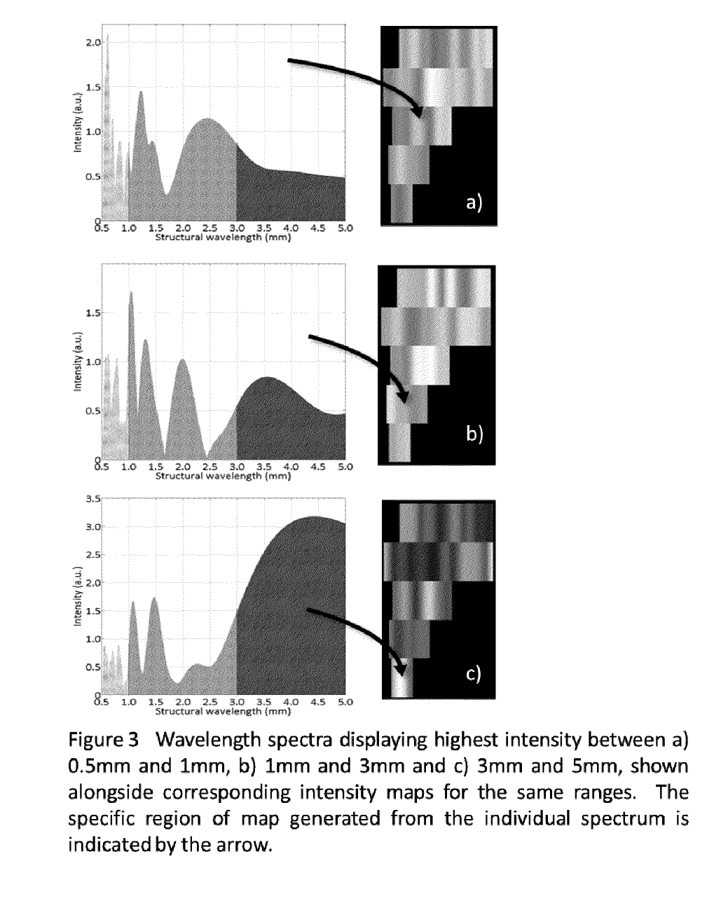

One adaptation is to base the selection of specific structural wavelength ranges to monitor, derive markers from, and map, on histological data of the disease (see FIG. 2), confocal microscopy, measurement of biological phantoms, or other such derived knowledge of the tissue changes expected in development of a particular disease. Mapping of quantities derived by various methods from the echo data obtained from a single or from an interleaved set of acquisition volumes—integrated intensity within a wavelength range, ratio of peak intensities, normalised intensities, ratio of integrated intensities, peak positions, comparing features in individual structural wavelength ranges to each other or to some derived spectral feature, or by use of statistical transform methods to subtract noise from the data, for example—can then be used for disease assessment and staging (see FIG. 3).

As previously described in the prior art, progression of liver disease entails the formation of fibrous tissue—rigid septa that can form interconnections. These interconnections, formed between collagenous fibers, form bridges between central veins, portal triads, and/or around hepatocytes. As these fibers form and connect, therefore, changes in the structural pattern of the tissue occur, progressing towards larger structural wavelengths representative of the separation between central veins and portal triads, i.e. vessel-to-vessel spacing and eventually lobule-to-lobule separation and/or central vein to central vein separation. This progression to longer wavelength textures is accompanied by attenuation in the shorter wavelength ranges.

In the liver, histology shows that the larger structure, i.e. general width of liver lobules or central vein-to-central vein separation, lie in the approximate range of 1 mm to 3 mm. The smaller features such as the separation between vessels (e.g. portal triad-to-portal triad) is on a scale of 1 mm or less. In addition to the normal dimensions of the liver lobules, there is the vascular structure in the liver (for example, the tertiary branches of the port triad structures or the smaller branches of the left and right hepatic veins) that lie within the 3 mm to 5 mm range, which are visible in conventional magnetic resonance imaging. As liver disease and fibrosis progresses, this vascular structure is altered. Therefore, the wavelength ranges of interests in detecting liver fibrosis cover the sub-millimeter range out to approximately 5 mm or 6 mm.

In healthy liver, the characteristic textural wavelength ranges of interest are produced by the healthy vessel-to-vessel separations (i.e. central vein-to-central vein and portal triad-to-portal triad in the classical liver lobule model). The repeating central vein-to-central vein pattern has a characteristic wavelength of ˜2 mm whilst the finer texture of the network of portal triads has a wavelength smaller than 1 mm. In the development of CLD, bridging fibrosis between portal triads is expected to progressively obscure the finer texture less than or approximately 1 mm arising from the somewhat regular pattern of portal triad-to-portal triad separation. Furthermore, as this bridging fibrosis progressively encases liver lobules by bridging between vessels on the periphery of the liver lobule it serves to enhance the larger textures above 1 mm.

To follow the advance of CLD, one could select these three disease-pertinent wavelength ranges to monitor for disease staging. Markers derived from the structural spectra data falling in these selected wavelength ranges would then be mapped using some form of quantification metric. For instance, as described in the prior art technique for mapping structural wavelength data across an areal cross-section, a different color or a monotone intensity to could be assigned to each of the ranges 0.56-1 mm, 1.00-3.00 mm, and 3.00 to 5.00 mm, and the color mapped at successive ROIs (regions of interest) along each interleaved acquisition volume. Once the wavelength ranges pertinent to disease development are identified based on knowledge of the disease, such as is provided by histology or other forms of tissue assessment, various markers derived from the structural wavelength data falling in these ranges can then be evaluated and mapped singly, or multiply on a single output map. Data within the targeted wavelength ranges can be used to derive these markers, or can be compared in some way to data falling outside these ranges on the structural wavelength spectra, such as might be done for normalisation.

In acquiring the data, the interleaved acquisition volumes would be positioned to cover a region of interest in the organ. In the liver, the interleaved acquisition volumes could be positioned in the right anatomical liver lobe, with the end of the volumes near or crossing the liver periphery. The right anatomical lobe could be chosen, because histology biopsies are acquired from the right liver lobe towards the liver periphery. However, the advantage of the technique is that it could be applied to the left hepatic liver lobe to assess liver disease in that lobe. In addition, as fibrotic invasion in an organ may follow a particular pattern across an organ, mapping the structural wavelengths, or other markers derived from the data, can be used as an ancillary assessment of disease. Thus, by targeting specific ranges of structural wavelengths, disease can be monitored both globally across an organ, and locally within a small ROI. After data acquisition, segmentation algorithms may be used to eliminate regions of the array of interleaved acquisition volumes that fall outside the organ under study.

Repeating data acquisition measurements at various times following administration of a contrast agent to follow the course of uptake and elimination of the agent for reasons such as distinguishing fibrosis from vasculature can be used to identify the various wavelength ranges that are represented most strongly in these two types of structure and to allow for correction of the data by subtraction of signal due to vasculature.

The cross-section dimensions of the acquisition volumes are chosen taking into account the wavelength ranges of interest: in the case of the liver dimensions described previously, the scale of the cross-sectional dimensions are on the order of a few mm on a side. The cross-sectional area is selected to be large enough to 1) allow sampling of several occurrences of the largest texture under study within each voxel, 2) increase signal to noise by sampling a larger voxel, and small enough to 3) allow localization of the textural information. The length of the internal volume is chosen to be appropriate to the size of the organ or anatomical area of interest; for liver the length of the selectively excited internal volumes has dimensions of tens of mm.

In an organ, echo data from multiple acquisition volumes are acquired to generate structural wavelength spectra from successive ROIs within the array. This acquisition can be performed in one breath hold in the liver, the acquisition time required being dependent on contrast and signal intensity for other diseases. Acquisition volumes in liver assessment can be arranged adjacent to each other with the opposing vertices aligned with the coronal plane to maximise coverage of the liver in one acquisition, as shown in FIG. 1. They can be located in either the anterior or the posterior part of the right hepatic lobe and are positioned to avoid intersection with the portal vein and the right hepatic vein.

In general, the location and orientation of the acquisition volumes can be adjusted to cover areas of interest in the organ. Multiple interleaved-volume acquisition series can be run to allow different orientations to be investigated in the organ of interest. Acquisition volumes aligned at differing angles through an organ can be used to evaluate anisotropy of fibrotic development to use as a disease marker.

This invention also includes the use of a normalization method to correct for the unavoidable differences in signal from patient to patient and to a lesser degree across an organ. These differences can arise from variations in the proximity of the coil to the organ, the type of coil used, and chemical differences in the liver tissue. To ensure that intensity measures from different studies can be compared, three basic methods of normalising the data from the various patients were developed: 1) normalising to the average MR signal intensity from the entire portion of the interleaved array falling within the organ boundary, 2) normalising to the average MR signal intensity from each separate acquisition volume of the array, within the organ boundary, or 3) normalising to the average MR signal intensity from each separate ROI defined along the acquisition volumes of the interleaved array, falling within the organ boundary. Two additional methods of normalizing the data between studies are to 1) normalize relative to noise levels in the data, 2) normalize by use of a calibration standard placed next to the patient, in proximity to the organ under study, and from which a signal is recorded during data acquisition.

Structural wavelength spectra localized to ROIs along each acquisition volume can be generated by windowing and filtering the normalized echo signals from each acquisition volume, and repeating this process for all segments using a sliding window. Then the average intensity in one or more wavelength bands, chosen as described below, determine the relative values of color, hue, or other indicator plotted on the map at the center of each filtered segment.

One application of the technique to liver disease may be to assist in the decision of whether or not to biopsy. Given that suspected Chronic Liver Disease CLD cases are routinely referred for MRI to rule out HCC, the addition of the technique to that scan would come at virtually no cost and provide significant added value. As such, and especially in the earlier stages of the disease, for which there is currently no good diagnostic, the technique may be used to replace biopsy altogether.

In addition to the application of the technique in liver disease, by targeted selection of structural wavelength ranges indicative of disease development, the technique can be applied to a range of fibrotic diseases. As in liver disease, the cross section of the acquisition volumes, number of volumes per array, the targeted organ or anatomy, contrast mechanisms, and specific echo-derived markers applied are disease specific and would be chosen to assess developing pathology particular to a disease state. Prior know-how, including histology, would inform the specific protocols. A partial list of these diseases is called out in the prior art section.

Thus the present invention has a number of aspects, which aspects may be practiced alone or in various combinations or sub-combinations, as desired. While certain preferred embodiments of the present invention have been disclosed and described herein for purposes of illustration and not for purposes of limitation, it will be understood by those skilled in the art that various changes in form and detail may be made therein without departing from the spirit and scope of the invention as defined by the full breadth of the following claims.

What is claimed is:

1. A method of assessing the development of fibrotic structure within an organ of a patient in response to disease of the patient comprising:
acquiring spatially-encoded MR echoes along an acquisition axis of a selectively-excited internal volume positioned within a targeted region in a patient while applying a magnetic field gradient;
analyzing the spatially-encoded MR echoes along an acquisition axis in the selectively-excited internal volume to yield a spectrum of textural wavelengths in a region of interest along a spatially-encoded axis of the internal volume as a marker of the disease;
assessing the development of fibrotic disease from the spectrum of textural wavelengths in the region of interest in the organ in comparison to known spectrums of textural wavelengths in a corresponding region of interest in the organ taken from the same or different patients.

2. The method of claim 1 wherein assessing the conditions or disease comprises analyzing selected ranges of the spectrum of textural wavelengths.

3. The method of claim 2 further comprising using a relative integrated intensity within the selected ranges of structural wavelength for assessing fibrotic development in that region of interest.

4. The method of claim 3 further comprising using the spectrum of textural wavelengths from a single acquisition volume or a set of acquisition volumes to generate a map of integrated intensity within selected wavelength ranges, displaying the integrated intensity as a number, a grey tone, or a color, for assessing disease state and monitoring progression in the organ.

5. The method of claim 3 further comprising using histology in the selection of the wavelength ranges of interest so as to provide best correlation with a specific disease.

6. The method of claim 1 further comprising using interleaved acquisition volumes to cover a targeted area of an organ, with analysis done at one or more regions of interest along the respective acquisition axes of the interleaved volumes to assess development of fibrotic disease in the organ.

7. The method of any one of claim 1, 2, 3 or 6 further comprising selecting the cross section of the acquisition volumes in the array taking into account the wavelength ranges of interest in the targeted disease.

8. The method of claim 1 further comprising generating and mapping at least one other marker from the MR echo data as part of assessing development of fibrotic structure within the organ.

9. The method of claim 8 wherein the other marker is selected from the group consisting of markers derived using the data in the structural wavelength ranges of interest for disease progression; a) taking the ratio of the height of various peaks within the spectrum of textural wavelengths to yield a marker, b) taking the ratio of integrated intensities of various peaks, or of normalized intensities of these peaks, c) comparing features in individual structural wavelength ranges to each other or to some derived spectral feature, or d) using statistical transform methods to subtract noise from the data.

10. The method of any of claim 1, 2, 3, 5, 6 or 9 further comprising repeating the method for multiple regions of interest to evaluate the variation in fibrotic structure across a region of interest within the organ using the spatial variation in integrated intensity in particular wavelength ranges to assess uniformity of disease in the organ.

11. The method of claim 10 further comprising repeating the method for multiple regions of interest to map the variation in fibrotic structure across the multiple regions of interest in the organ.

12. The method of any one of claim 1, 2, 3, 6, or 11 further comprising repeating MR scans over the course of disease development or treatment for assessment of response to therapy.

13. The method of claim 12 further comprising using segmentation to eliminate the portions of the interleaved array that fall outside the organ of interest.

14. The method of claim 1 further comprising using an endogenous MR contrast or administration of an exogenous MR contrast agent to the patient.

15. The method of claim 14 further comprising repeating the method at various times following administrating the exogenous MR contrast agent to follow the course of uptake and elimination of contrast agent.

16. The method of claim 15 wherein the method is repeated to distinguish fibrotic structure from vasculature to identify wavelength ranges that are represented most strongly in fibrotic structure development from vasculature and to allow for correction of the spatially-encoded MR echoes by subtraction of spatially-encoded MR echoes due to vasculature.

17. The method of claim 1 further comprising using segmentation to eliminate the portions of the interleaved array that fall outside the organ of interest.

18. The method of claim 1 comprising using the method for assessing development of fibrotic structure of liver, lung, myocardiac fibrosis, muscle fibrosis, cystic fibrosis, pancreatic fibrosis.

19. The method of claim 1 comprising practicing the method on multiple sets of interleaved or single internal volumes to determine variation in fibrotic structure at multiple different planes in an organ.

20. The method of any one of claim 1, 2, 3, 6, 14, 15 or 19 wherein the method is repeated for regions of interest across an organ, and assessing the disease stage type.

21. The method of claim 1 further comprising using alternate markers derived from defined structural wavelength ranges that are particular to a disease process, mapped as color, hue, numerical value, or other metric indicating the magnitude of the marker, such as density of an icon.

22. The method of claim 1 further comprising normalizing the echo signals to correct for differences in signal intensity, thereby allowing comparisons of the assessment between patients.
